SEQUENCE LISTING (4 sequences)

SEQ ID NO: 1 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 145 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTR

SEQ ID NO: 3 - protein, 270 aa, Homo sapiens
AETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - protein, 235 aa, Homo sapiens
VVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

(19) United States
(12) Patent Application Publication
(10) Pub. No.: US 2026/0258152 A1
(43) Pub. Date: Sep. 3, 2026

(54) COMPOSITIONS AND METHODS FOR TREATING FACTOR IX DEFICIENCY

(71) Applicant: The Children's Hospital of Philadelphia, Philadelphia, PA (US)

(72) Inventor: Benjamin J. Samelson-Jones, Wynnewood, PA (US)

(21) Appl. No.: 18/832,149

(22) PCT Filed: Jan. 31, 2023
(86) PCT No.: PCT/US23/61626

§ 371 Date: Jul. 23, 2024

Related U.S. Application Data
(60) Provisional application No. 63/304,984, filed on Jan. 31, 2022.

(51) Int. Cl. C07K 16/36; A61K 39/00; A61P 7/04; C12Q 1/6883

(52) U.S. Cl. CPC C07K 16/36; A61P 7/04; C12Q 1/6883; A61K 2039/505; C07K 2317/24; C07K 2317/31; C12Q 2600/156

(57) ABSTRACT

Compositions and methods for treating hemophilia B are disclosed.

This application claims priority under 35 U.S.C. § 119 (e) to U.S. Provisional Patent Application No. 63/304,984, filed Jan. 31, 2022. The foregoing application is incorporated by reference herein.

### FIELD OF THE INVENTION

The present invention relates to the fields of medicine and hematology. More specifically, the invention provides novel methods for treating Factor IX deficiency.

### BACKGROUND OF THE INVENTION

Several publications and patent documents are cited throughout the specification in order to describe the state of the art to which this invention pertains. Each of these citations is incorporated herein by reference as though set forth in full.

In response to vascular injury such as a cut, coagulation enzymes are activated in a stepwise manner, ultimately resulting in the formation of a blood clot at the site of injury. Thrombin is generated from its inactive precursor prothrombin in the final step of this cascade and subsequently produces the fibrous clot. Activated Factor IX (FIXa) is a key component of this system as it is the serine protease of the intrinsic Xase complex, which also comprises the co-factor activated Factor VIII (FVIIIa). This enzyme complex, assembled on cells with exposed anionic membranes, rapidly converts Factor X to activated Factor X (FXa). FXa and its co-factor, activated Factor V (FVa), form prothrombinase, the enzyme complex that activates thrombin.

The importance of Factor IX is reflected by the occurrence of the bleeding disorder hemophilia B in individuals carrying mutations in the Factor IX gene. In bleeding disorders such as hemophilia B, a defect or deficiency of Factor IX results in inadequate FXa generation and, therefore, inadequate thrombin formation. Protein replacement therapy with intravenously delivered plasma-derived or recombinant Factor IX is the standard of care for hemophilia B patients. While this therapy is efficient, this therapy requires frequent (e.g., 1-3 times per week) intravenous administration of large amounts of Factor IX due to the short half-life of the protein (Butterfield, et al., Mol. Ther. (2020) 28 (3): 997-1015). Therefore, there is an obvious need for improved therapeutic methods.

### SUMMARY OF THE INVENTION

In accordance with the present invention, methods for treating, inhibiting, and/or preventing treating Factor IX deficiency and/or hemophilia B in a subject are provided. In certain embodiments, the methods comprise administering a therapeutically effective amount of a FVIII mimicking bispecific antibody to the subject. In certain embodiments, the subject comprises a Factor IX mutant or variant which causes HB. In certain embodiments, the subject comprises a Factor IX mutant or variant which has a disrupted or reduced interaction or binding affinity between FIXa and FVIIIa and/or the Factor IX mutant has reduced allosteric activation by FVIIIa (e.g., compared to wild-type). In certain embodiments, the Factor IX mutant comprises a mutation within the FVIIIa binding site of FIX. In certain embodiments, the Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397. In certain embodiments, the Factor IX mutant comprises a mutation at R338, E387, 1397, and/or

G93. In certain embodiments, the Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, I344F, 1344N, 1344P, 1344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T. In certain embodiments, the Factor IX mutant comprises a mutation selected from the group consisting of R338P, E387K, I397T, and G93S. In certain embodiments, the FVIII mimicking bispecific antibody binds Factor IXa and Factor X. In certain embodiments, the FVIII mimicking bispecific antibody is emicizumab or mim8. In certain embodiments, the method further comprises determining the subject comprises a Factor IX mutant prior to treatment, optionally by obtaining a biological sample from the subject.

In accordance with the present invention, methods for increasing the coagulation of blood are provided. In certain embodiments, the methods comprise contacting the blood with a FVIII mimicking bispecific antibody. In certain embodiments, the subject comprises a Factor IX mutant or variant which causes HB. In certain embodiments, the blood comprises a Factor IX mutant which disrupts the interaction between FIXa and FVIIIa (e.g., compared to wild-type). In certain embodiments, Factor IX mutant has reduced allosteric activation by FVIIIa (e.g., compared to wild-type). In certain embodiments, the Factor IX mutant comprises a mutation within the FVIIIa binding site of FIX. In certain embodiments, the Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397. In certain embodiments, the Factor IX mutant comprises a mutation at R338, E387, 1397, and/or G93. In certain embodiments, the Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, I344F, 1344N, I344P, I344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T. In certain embodiments, the Factor IX mutant comprises a mutation selected from the group consisting of R338P, E387K, I397T, and G93S. In certain embodiments, the FVIII mimicking bispecific antibody binds Factor IXa and Factor X. In certain embodiments, the FVIII mimicking bispecific antibody is emicizumab or mim8.

### BRIEF DESCRIPTION OF THE DRAWINGS

FIG. 1A provides a graph of the measured FIX activity of the indicated variants with or without 300 nM emicizumab. Left: Recombinant FIX variants causing hemophilia B were expressed from mammalian cells. Conditioned media was assayed for FIX activity with and without 300 nM emicizumab. Right: FIX activity from patient plasma before or after 300 nM emicizumab was added to sample. Horizontal lines demarcate severe, moderate, and mild hemophilia. Values are normalized to FIX-WT. Errors bars are SEM (n=3). FIG. 1B provides a log scale graph of the measured FIX activity of the indicated variants with or without 300 nM emicizumab.

FIG. 2 provides a graph of the amplitude in a rotational thromboelastometry (ROTEM) assay with modified whole blood from a patient with moderate hemophilia B (HB) due to FIX I397T or with modified whole blood from a patient with moderate HB due to FIX I397T treated with recombinant FIX or 300 nm emicizumab (emi).

FIG. 3A provides graphs of thrombin generation by dysfunctional HB-causing FIX variants with (*) and without 300 nM emicizumab. FIG. 3B provides a graph of the quantification of peak thrombin generation by dysfunctional HB-causing FIX variants with (dark grey) and without (light grey) 300 nM emicizumab. Error bars represent standard deviation. UC: negative control of unconditioned media.

FIG. 4A provides a graph of the clotting time of HB FIX I397T and HB plasma in the presence of the indicated amounts of emicizumab. Each point represents the mean of four measurements with error bars indicating standard deviation. FIG. 4B provides a graph of FIX activity measured using a one-stage activated partial thromboplastin time (aPTT) assay of FIX I397T or HB plasma with and without 300 nM emicizumab.

FIG. 5 provides graphs showing the clotting activity of rescuable hemophilia B causing FIX variants with and without FVIII mimetic. HB-causing variants were recombinantly expressed in HEK293 cells. FIX activity with 0 nM (light gray bars) or 300 nM (dark grey bars) emicizumab was determined with a modified one-stage assay based on standard curve with wild-type FIX with the same concentration of emicizumab. Rescuable variants (top) have ≥2 fold increase in FIX activity with the addition of emicizumab. Control FIX variants (bottom) include wild-type (WT), non-deleterious polymorphism (A148T), catalytically inert (S365A), hyperactive R338L, and unconditioned media (UC). Bars represent mean activity of >3 independent transfections with error bars are SEM.

FIGS. 6A and 6B provide graphs of thrombin generation of rescuable hemophilia B-causing FIX variants with and without FVIII mimetic. Thrombin generation was measured for FIX variants (right peaks) and with 300 nM emicizumab (left peaks).

### DETAILED DESCRIPTION OF THE INVENTION

The recent advent of bispecific antibody factor VIII (FVIII)-mimetics has revolutionized the clinical care of hemophilia A (HA, FVIII deficiency). Activated factor IX (FIXa) and FVIIIa are responsible for the proteolytic activation of factor X (FX), the rate limiting step of clot formation. Herein, it is shown that patients with hemophilia B (HB, FIX deficiency) caused by mutations that result in dysfunctional FIX/FIXa activity due to disrupted FIXa-FVIIIa interactions have improvement in their hemostatic function using bispecific antibody FVIII-mimetics. At least two types of disrupted FIXa-FVIIIa interactions can benefit from the bispecific antibody FVIII-mimetic: 1) decreases in FVIIIa binding affinity to FIXa and 2) decreases in FVIIIa-induced allosteric activation of FIXa. Herein, the missense variant I397T, which disrupts the FVIIIa-induced allosteric activation of FIXa, was tested among other FIX mutations. The baseline FIX activity levels of FIX I397T are 2-3% normal. It is shown herein that emicizumab has a dose-dependent improvement in the clotting time including normalizing this clotting time at 300 nM, which is the current therapeutic concentration for HA patients. In contrast, emicizumab has no effect on commercial FIX-deficient plasma. It is also shown herein that the measured FIX activity with 300 nM emicizumab for FIX I397T is 12% normal. This level of FIX activity is well within the mild HB range and is associated with a complete abrogation of spontaneous bleeds.

Enhancement of FIX activity of unscreened HB plasma has been evaluated (Ogiwara et al., Res. Prac. Thrombosis Haemostasis (2017) 1 (S1): 749; Yada et al., Arterioscler. Thromb. Vasc. Biol. (2020) 40:1148-1154). Ogiwara et al. tested the clotting time of samples from 17 HB patients in the presence of emicizumab and found “large variations” from “no response [to] ~60%.” Notably, Ogiwara et al. teach that plasma from HB patients with a dysfunctional FIX were in the “poor response group.”

In stark and direct contrast to Ogiwara et al., it is shown herein that dysfunctional FIX variants which disrupt the FIXa-FVIIIa interaction can be rescued with emicizumab. Indeed, the clotting activity of dysfunctional FIX variants with a disrupted FIXa-FVIIIa interaction is exquisitely sensitive to FVIII-mimicking bispecific antibody rescue.

Factor IX circulates in plasma as an inactive zymogen (57 kDa) at a concentration of approximately 90 nM. Factor IX is initially synthesized in the liver harboring a ~40 amino acid pre-pro-leader sequence which is removed upon secretion of the protein. Upon vascular damage, Factor IX is activated to FIXa following cleavage of two bonds by either the Factor VIIa/tissue factor (TF) complex or Factor XIa-releasing a 35-residue activation peptide. Free FIXa is rapidly eliminated by reactions with circulating serpins, most notably antithrombin III (ATIII).

Gene ID: 2158 and GenBank Accession Nos. NM_000133.3 and NP_000124.1 provide examples of the amino acid and nucleotide sequences of wild-type human Factor IX preproprotein. An example of the amino acid sequence of the human Factor IX preproprotein is:

| (SEQ ID NO: 1) | |
|---|---|
| 1 | MQRVNMIMAE SPGLITICLL GYLLSAECTV FLDHENANKI LNRPKRYNSG KLEEFVQGNL |
| 61 | ERECMEEKCS FEEAREVFEN TERTTEFWKQ YVDGDQCESN PCLNGGSCKD DINSYECWCP |
| 121 | FGFEGKNCEL DVTCNIKNGR CEQFCKNSAD NKVVCSCTEG YRLAENQKSC EPAVPFPCGR |
| 181 | VSVSQTSKLT RAETVFPDVD YVNSTEAETI LDNITQSTQS FNDFTRVVGG EDAKPGQFPW |
| 241 | QVVLNGKVDA FCGGSIVNEK WIVTAAHCVE TGVKITVVAG EHNIEETEHT EQKRNVIRII |
| 301 | PHHNYNAAIN KYNHDIALLE LDEPLVLNSY VTPICIADKE YTNIFLKFGS GYVSGWGRVF |
| 361 | HKGRSALVLQ YLRVPLVDRA TCLRSTKFTI YNNMFCAGFH EGGRDSCQGD SGGPHVTEVE |
| 421 | GTSFLTGIIS WGEECAMKGK YGIYTKVSRY VNWIKEKTKL T |

The bolded and underlined amino acids are amino acids discussed herein. The Factor IX prepropetide comprises a signal peptide from amino acids 1-28 and a propeptide sequence from amino acids 29-46. Numbering of amino acids for FIX/FIXa begin at position 47. The cleavage of the propeptide yields a protein with a new terminus sequence of Tyr-Asn-Ser. Factor IX is also cleaved by the FVIIa/TF complex or FXIa into a mature two-chain form (light and heavy) at an arginyl-alanine peptide bond to generate the Factor IX zymogen. The two chains are linked via a disulfide bond. An example of the amino acid sequence of the human Factor IX light chain is (bolded and underlined amino acids are amino acids discussed herein):

(SEQ ID NO: 2)
YNSG KLEEFVQGNL ERECMEEKCS FEEAREVFEN TERTTEFWKQ YVDGDQCESN
PCLNGGSCKD DINSYECWCP FGFEGKNCEL DVTCNIKNGR CEQFCKNSAD
NKVVCSCTEG YRLAENQKSC EPAVPFPCGR VSVSQTSKLT R

An example of the amino acid sequence of the human Factor IX heavy chain is (bolded and underlined amino acids are amino acids discussed herein):

(SEQ ID NO: 3)
AETVFPDVD YVNSTEAETI LDNITQSTQS FNDFTRVVGG EDAKPGQFPW
QVVLNGKVDA FCGGSIVNEK WIVTAAHCVE TGVKITVVAG EHNIEETEHT
EQKRNVIRII PHHNYNAAIN KYNHDIALLE LDEPLVLNSY VTPICIADKE
YTNIFLKFGS GYVSGWGRVF HKGRSALVLQ YLRVPLVDRA TCLRSTKFTI
YNNMFCAGFH EGGRDSCQGD SGGPHVTEVE GTSFLTGIIS WGEECAMKGK
YGIYTKVSRY VNWIKEKTKL T

Factor IX is activated by the cleavage of the 35 amino acid activation peptide at an arginyl-valine peptide bond by the FVIIa/TF complex or FXIa to yield a new amino-terminal sequence. An example of the amino acid sequence of the human Factor IXa light chain is (bolded and underlined amino acids are amino acids discussed herein):

(SEQ ID NO: 2)
YNSG KLEEFVQGNL ERECMEEKCS FEEAREVFEN TERTTEFWKQ YVDGDQCESN
PCLNGGSCKD DINSYECWCP FGFEGKNCEL DVTCNIKNGR CEQFCKNSAD
NKVVCSCTEG YRLAENQKSC EPAVPFPCGR VSVSQTSKLT R

An example of the amino acid sequence of the human Factor IXa heavy chain is (bolded and underlined amino acids are amino acids discussed herein):

(SEQ ID NO: 4)
VVGG EDAKPGQFPW QVVLNGKVDA FCGGSIVNEK WIVTAAHCVE TGVKITVVAG
EHNIEETEHT EQKRNVIRII PHHNYNAAIN KYNHDIALLE LDEPLVLNSY
VTPICIADKE YTNIFLKFGS GYVSGWGRVF HKGRSALVLQ YLRVPLVDRA
TCLRSTKFTI YNNMFCAGFH EGGRDSCQGD SGGPHVTEVE GTSFELTGIIS
WGEECAMKGK YGIYTKVSRY VNWIKEKTKL T

Notably, the above proteolytic cleavage events may be imprecise, thereby leading to addition or loss of amino acids (e.g., 1, 2, 3, or more amino acids) at the cleavage sites. Nucleic acid molecules which encode Factor IX and FIXa can be readily determined from the provided amino acid sequences.

The instant invention encompasses methods of inhibiting, treating, and/or preventing hemophilia B. In certain embodiments, the method comprises administering to a subject in need thereof a therapeutically effective amount of a FVIII mimicking (mimetic) bispecific antibody. In certain embodiments, the FVIII mimicking bispecific antibody comprises a first binding domain (e.g., an antigen binding antibody fragment, such as a Fab arm) which binds FIXa and/or FIX and a second binding domain (e.g., an antigen binding antibody fragment, such as a Fab arm) which binds FX and/or FXa. The FVIII mimicking bispecific antibody, by binding both FIXa and FX brings the two proteins into proximity such that FIXa activates FX to FXa. In certain embodiments, the FVIII mimicking bispecific antibody comprises an Fc region. In certain embodiments, the FVIII mimicking bispecific antibody comprises an anti-FIXa heavy chain, an anti-FX heavy chain, and two light chains (optionally identical or different). In certain embodiments, the FVIII mimicking bispecific antibody is emicizumab (ACE910; HEMLIBRA®; Genentech; DrugBank Accession Number DB13923; Lenting, et al. Blood (2017) 130 (23): 2463-2468; Walsh, et al., J. Managed Care Med., 22 (2): 65-69). In certain embodiments, the FVIII mimicking bispecific antibody is mim8 (Novo Nordisk; Kjellev et al., Blood (2019) 134 (Supplement_1): 96; Ostergaard et al., Blood (2021) 138 (14): 1258-1268). In certain embodiments, the FVIII mimicking bispecific antibody is described in U.S. Patent Application Publication No. 2020/0148787 or U.S. Pat. No. 10,759,870 or 11,150,254 (each incorporated herein by reference).

In certain embodiments, the subject being treated has a mutation in FIX. In certain embodiments, the subject comprises a Factor IX mutant or variant which causes HB. In certain embodiments, the FIX mutation disrupts the interaction between FIXa and FVIIIa. In certain embodiments, the FIX mutation lowers the binding affinity of FIXa for FVIIIa (e.g., compared to wild-type FIX/FIXa). In certain embodiments, the FIX mutation decreases the FVIIIa-induced allosteric activation of FIXa (e.g., compared to wild-type FIX/FIXa). In certain embodiments, the FIX mutation is located within the FVIIIa binding site of FIX.

In certain embodiments, the FIX mutation is at N2, S3, K5, L6, F25, D47, N67, E78, E83, N92, G93, R180, V182, A233, D332, R333, R338, T340, K341, I344, M348, G356, P368, T376, E387, C389, and/or I397. In certain embodiments, the FIX mutation is at N2, S3, K5, L6, F25, D47, N67, E78, E83, N92, G93, V182, A233, D332, R333, R338, T340, K341, I344, M348, G356, P368, T376, E387, C389, and/or I397. In certain embodiments, the FIX mutation is at N2, S3, K5, L6, F25, D47, N67, E78, E83, N92, G93, V182, D332, R333, R338, T340, K341, I344, M348, G356, P368, T376, E387, and/or I397. In certain embodiments, the FIX mutation is at N2, S3, K5, L6, F25, D47, N67, N92, V182, D332, R333, R338, K341, I344, G356, T376, E387, and/or I397. In certain embodiments, the FIX mutation is at K5, L6, D47, D332, R333, R338, P368, E387, and/or I397. In certain embodiments, the FIX mutation is at C389, A233, R180, R338, E387, 1397, and/or G93. In certain embodiments, the FIX mutation is at R338, E387, 1397, and/or G93. In certain embodiments, the FIX mutation is at R338, E387, and/or I397.

In certain embodiments, the mutation at N2 is N2D, N2Y, or N2I, particularly N2I. In certain embodiments, the mutation at S3 is S3P. In certain embodiments, the mutation at K5 is K5E. In certain embodiments, the mutation at L6 is L6S. In certain embodiments, the mutation at F25 is F25C or F25S. In certain embodiments, the mutation at D47 is D47N, D47H, D47E, or D47G, particularly D47E or D47G. In certain embodiments, the mutation at N67 is N67K. In certain embodiments, the mutation at E78 is E78K. In certain embodiments, the mutation at E83 is E83K. In certain embodiments, the mutation at N92 is N92H, N92K, or N92S, particularly N92K or N92S. In certain embodiments, the mutation at G93 is G93D, G93S, G93R, G93K, G93N, particularly G93D or G93S, more particularly G93S. In certain embodiments, the mutation at R180 is R180W, R180Q, R180G, R180P, or R180L, particularly R180W or R180Q. In certain embodiments, the mutation at V182 is V182L, V182F, V182A, or V182G, particularly V182L. In certain embodiments, the mutation at A233 is A233T. In certain embodiments, the mutation at D332 is D332Y. In certain embodiments, the mutation at R333 is R333G, R333L, R333P, or R333Q, particularly R333L, R333P, or R333Q. In certain embodiments, the mutation at R338 is R338P. In certain embodiments, the mutation at R338 is not R338L. In certain embodiments, the mutation at T340 is T340I or T340R, particularly T340R. In certain embodiments, the mutation at K341 is K341N or K341E, particularly K341E. In certain embodiments, the mutation at 1344 is 1344F, 1344N, 1344P, 1344S, or I334T. In certain embodiments, the mutation at M348 is M348V, M348R, M348I, or M348K, particularly M348I or M348K. In certain embodiments, the mutation at G356 is G356R or G356E, particularly G356R. In certain embodiments, the mutation at P368 is P368S, P368L, P368R, P368H, or P368T, particularly P368H or P368T. In certain embodiments, the mutation at T376 is T376N. In certain embodiments, the mutation at E387 is E387A, E387G, or E387K, particularly E387G or E387K. In certain embodiments, the mutation at C389 is C389Y, C389G, C389S, or C389R, particularly C389Y. In certain embodiments, the mutation at I397 is I397L or I397T, particularly I397T.

The methods of the instant invention may further comprise determining if the subject to be treated has a mutant FIX. In certain embodiments, the FIX mutation lowers the binding affinity of FIXa for FVIIIa (e.g., compared to wild-type FIX/FIXa). In certain embodiments, the FIX mutation decreases the FVIIIa-induced allosteric activation of FIXa (e.g., compared to wild-type FIX/FIXa). In certain embodiments, the FIX mutation is located within the FVIIIa binding site of FIX. In certain embodiments, the FIX mutation is at one of locations set forth herein or is one of the FIX mutations set forth herein. In certain embodiments, the method further comprises obtaining a biological sample (e.g., blood) from the subject.

Whether a subject has a mutant FIX can be determined by any method known in the art. In certain embodiments, a nucleic acid encoding FIX from a subject may be analyzed. For example, all or part of a nucleic acid sequence encoding FIX (e.g., gene, mRNA, etc.) may be determined or sequenced from a biological sample from the subject and then it can be determined whether the encoded for FIX comprises any mutations compared to wild-type FIX. In certain embodiments, the FIX protein from a subject may be analyzed. For example, all or part of the amino acid sequence from the FIX from the subject may be determined and compared to the wild-type FIX sequence. Binding assays or activation assays may also be performed with the FIX from the subject (e.g., isolated from the subject or recombinantly produced) may also be performed to determine if the FIX/FIXa from the subject has a lower binding affinity for FVIIIa (e.g., compared to wild-type FIX/FIXa) or has a decreased FVIIIa-induced allosteric activation (e.g., compared to wild-type FIX/FIXa).

The instant invention also encompasses methods of increasing the coagulation of blood (e.g., compared to the blood without treatment or to wild-type blood). For example, the method may decrease the clot time of the blood (e.g., compared to the blood clot time without treatment or to wild-type blood). In certain embodiments, the method decreases the clot time of the blood to less than a minute, particularly less than about 40 seconds. In certain embodiments, the method decreases the clot time of the blood to about normal clot time for blood. The method may be performed in vitro or in vivo. In certain embodiments, the method comprises contacting the blood with a FVIII mimicking bispecific antibody. In certain embodiments, the FVIII mimicking bispecific antibody comprises a first binding domain (e.g., an antigen binding antibody fragment, such as a Fab arm) which binds FIXa/FIX and a second binding domain (e.g., an antigen binding antibody fragment, such as a Fab arm) which binds FX/FXa. The FVIII mimicking bispecific antibody, by binding both FIXa and FX brings the two proteins into proximity such that FIXa activates FX to FXa. In certain embodiments, the FVIII mimicking bispecific antibody comprises an Fc region. In certain embodiments, the FVIII mimicking bispecific antibody comprises an anti-FIXa heavy chain, an anti-FX heavy chain, and two light chains (optionally identical or different). In certain embodiments, the FVIII mimicking bispecific antibody is emicizumab (ACE910; HEMLIBRA®; Genentech; DrugBank Accession Number DB13923; Lenting, et al. Blood (2017) 130 (23): 2463-2468; Walsh, et al., J. Managed Care Med., 22 (2): 65-69). In certain embodiments, the FVIII mimicking bispecific antibody is mim8 (Novo Nordisk; Kjellev et al., Blood (2019) 134 (Supplement_1): 96; Ostergaard et al., Blood (2021) 138 (14): 1258-1268). In certain embodiments, the FVIII mimicking bispecific antibody is described in U.S. Patent Application Publication No. 2020/0148787 or U.S. Pat. No. 10,759,870 or 11,150,254 (each incorporated herein by reference).

In certain embodiments, the blood comprises a mutant FIX. In certain embodiments, the Factor IX mutant or variant which causes HB. In certain embodiments, the FIX mutation disrupts the interaction between FIXa and FVIIIa. In certain embodiments, the FIX mutation lowers the binding affinity of FIXa for FVIIIa (e.g., compared to wild-type FIX/FIXa). In certain embodiments, the FIX mutation decreases the FVIIIa-induced allosteric activation of FIXa (e.g., compared to wild-type FIX/FIXa). In certain embodiments, the FIX mutation is located within the FVIIIa binding site of FIX. In certain embodiments, the FIX mutation is at one of locations set forth herein or is one of the FIX mutations set forth herein.

In accordance with the instant invention, methods of determining whether a subject (e.g., a hemophilia B subject) can be treated with a FVIII mimicking bispecific antibody are provided. In certain embodiments, the comprises obtaining blood or a fraction thereof (e.g., whole blood, plasma, purified or isolated FIX, etc.) from the subject. In certain embodiments, the method comprises determining if the subject has a mutant FIX, as described hereinabove (e.g., by sequencing). In certain embodiments, the method comprises contacting blood or a fraction thereof (e.g., plasma, purified or isolated FIX, etc.) with a FVIII mimicking bispecific antibody and determining if FIX activity is increased (e.g., compared to the absence of the FVIII mimicking bispecific antibody), wherein an increase in FIX activity indicates the subject can be treated with a FVIII mimicking bispecific antibody. In certain embodiments, FIX activity is measured by a clotting assay (e.g., an aPTT assay). In certain embodiments, FIX activity is measured by rotational thromboelastometry. In certain embodiments, FIX activity is measured by thrombin generation.

The FVIII mimicking bispecific antibody may be administered as part of a composition with a pharmaceutically acceptable carrier. The compositions of the instant invention may be conveniently formulated for administration with any carrier, particularly any pharmaceutically acceptable carrier(s). Except insofar as any conventional carrier is incompatible with the agents to be administered, its use in the pharmaceutical composition is contemplated. For example, the active agents may be formulated with an acceptable medium such as sterile liquid, water, aqueous solutions, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the active agents in the chosen medium may be varied and the medium may be chosen based on the desired route of administration of the pharmaceutical preparation. Except insofar as any conventional media or agent is incompatible with the active agents to be administered, its use in the pharmaceutical preparation is contemplated.

The compositions of the present invention can be administered by any suitable route, for example, by infusion, injection or other modes of administration such as controlled release devices. In certain embodiments, the composition is delivered by injection. In certain embodiments, the composition is delivered by intravenous injection. In certain embodiments, the composition is delivered subcutaneously. The compositions of the instant invention may be directly administered or applied to the site of bleeding (e.g., by injection). In general, pharmaceutical compositions and carriers of the present invention comprise, among other things, pharmaceutically acceptable buffers, diluents, liquids (such as water, saline, glycerol, sugars and ethanol), preservatives, stabilizing agents, solubilizers, emulsifiers, wetting agents, pH buffering substances adjuvants and/or carriers. Such compositions can include diluents of various buffer content (e.g., saline, Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., benzyl alcohol) and bulking substances (e.g., lactose, mannitol). For example, the preparation can be formulated with a buffer containing salts, such as NaCl, CaCl₂), and amino acids, such as glycine and/or lysine, and in a pH range from 6 to 8. The pharmaceutical compositions may be formulated in aqueous solutions (e.g., physiologically compatible buffers). Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. The compositions of the invention may also be incorporated into particulate preparations of polymeric compounds such as polylactic acid, polyglycolic acid, etc., or into liposomes or micelles, or mixed with phospholipids or micelles to increase stability. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E. W. Martin (Mack Pub. Co., Easton, Pa.) and “Remington: The Science and Practice Of Pharmacy” by Alfonso R. Gennaro (Lippincott Williams & Wilkins) which are herein incorporated by reference. The pharmaceutical composition of the present invention can be prepared, for example, in liquid form, deep-frozen, or can be in dried powder form (e.g., lyophilized). In a particular embodiment, when the preparation is stored in lyophilized form, it may be dissolved into a visually clear solution using an appropriate reconstitution solution prior to administration.

The compositions described herein will generally be administered to a patient as a pharmaceutical preparation. The term “patient” or “subject”, as used herein, refers to human or animal subjects. The compositions of the instant invention may be employed therapeutically, under the guidance of a physician.

The dose and dosage regimen of the compositions according to the invention that are suitable for administration to a particular patient may be determined by a physician considering the patient's age, sex, weight, general medical condition, and the specific condition for which the active agent is being administered and the severity thereof (e.g., the severity of the bleeding). The physician may also take into account the route of administration, the pharmaceutical carrier, and the particular agent's biological activity.

Selection of a suitable pharmaceutical preparation will also depend upon the mode of administration chosen. For example, the compositions of the invention may be administered by direct injection to a desired site. In this instance, a pharmaceutical preparation comprises the active agents of the instant invention dispersed in a medium that is compatible with the site of injection. The compositions of the instant invention may be administered by any method. For example, the compositions can be administered, without limitation, intravenously. Pharmaceutical preparations for injection are known in the art. If injection is selected as a method for administering the compositions, steps must be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the composition may be determined by evaluating the toxicity of the molecules or cells in animal models. Various concentrations of active agents in pharmaceutical preparations may be administered to mice or other animal models, and the minimal and maximal dosages may be determined based on the beneficial results and side effects observed as a result of the treatment. Appropriate dosage unit may also be determined by assessing the efficacy of the treatment in combination with other standard drugs. The dosage units of the compositions of the instant invention may be determined individually or in combination with each treatment according to the effect detected.

The invention includes, but is not limited to, the embodiments of the following numbered paragraphs:

1. A method of treating or inhibiting hemophilia B in a subject in need thereof comprising administering a therapeutically effective amount of a Factor VIII (FVIII) mimicking bispecific antibody to said subject, wherein said subject comprises a Factor IX (FIX) mutant.

2. The method of paragraph 1, wherein activated FVIII (FVIIIa) has decreased binding affinity for the activated FIX (FIXa) mutant compared to wild-type FIXa and/or the FIXa mutant has decreased FVIIIa-induced allosteric activation compared to wild-type FIXa.

3. The method of paragraph 1 or 2, wherein said FIX mutant comprises a mutation within the FVIIIa binding site of FIX.

4. The method of any one of paragraphs 1-3, wherein said Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397.

5. The method of any one of paragraphs 1-4, wherein said Factor IX mutant comprises a mutation at N2, S3, K5, L6, F25, D47, N67, E78, E83, N92, G93, V182, D332, R333, R338, T340, K341, I344, M348, G356, P368, T376, E387, and/or I397.

6. The method of any one of paragraphs 1-5, wherein said FIX mutant comprises a mutation at C389, A233, R180, R338, E387, 1397, and/or G93.

7. The method of any one of paragraphs 1-6, wherein said FIX mutant comprises a mutation at R338, E387, 1397, and/or G93.

8. The method of any one of paragraphs 1-7, wherein said FIX mutant comprises a mutation at R338, E387, and/or I397.

9. The method of any one of paragraphs 1-8, wherein said Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, 1344F, 1344N, 1344P, 1344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T.

10. The method of any one of paragraphs 1-9, wherein said Factor IX mutant comprises a mutation selected from the group consisting of N2I, S3P, K5E, L6S, F25C, F25S, D47E, D47G, N67K, E78K, E83K, N92K, N92S, G93S, V182L, D332Y, R333L, R333P, R333Q, R338P, T340R, K341E, 1344F, 1344N, 1344P, 1344S, 1334T, M348I, M348K, G356R, P368H, P368T, T376N, E387A, E387G, E387K, and I397T.

11. The method of any one of paragraphs 1-10, wherein said FIX mutant comprises a mutation selected from the group consisting of C389Y, C389G, C389S, C389R, A233T, R180W, R180Q, R180G, R180P, R180L, R338P, E387A, E387G, E387K, I397T, and G93S.

12. The method of any one of paragraphs 1-11, wherein said Factor IX mutant comprises a mutation selected from the group consisting of R338P, E387K, I397T, and G93S.

13. The method of any one of paragraphs 1-12, wherein said FIX mutant comprises a mutation selected from the group consisting of R338P, E387K, and I397T.

14. The method of any one of paragraphs 1-13, wherein said FVIII mimicking bispecific antibody binds Factor IXa and Factor X.

15. The method of any one of paragraphs 1-14, wherein said FVIII mimicking bispecific antibody is emicizumab.

16. The method of any one of paragraphs 1-15, wherein said FVIII mimicking bispecific antibody is mim8.

17. The method of any one of paragraphs 1-16, further comprising determining said subject comprises said Factor IX mutant prior to administering said FVIII mimicking bispecific antibody.

18. The method of paragraph 17, wherein determining said subject comprises said Factor IX mutant comprises at least partially sequencing a nucleic acid encoding Factor IX from said subject.

19. The method of any one of paragraphs 1-18, further comprising obtaining a biological sample from said subject.

20. A method for increasing blood coagulation comprising contacting blood with a Factor VIII (FVIII) mimicking bispecific antibody, wherein said blood comprises a Factor IX (FIX) mutant.

21. The method of paragraph 20, wherein activated FVIII (FVIIIa) has decreased binding affinity for the activated FIX (FIXa) mutant compared to wild-type FIXa and/or the FIXa mutant has decreased FVIIIa-induced allosteric activation compared to wild-type FIXa.

22. The method of paragraph 20 or 21 which is an in vitro method.

23. The method of any one of paragraphs 20-22, wherein said Factor IX mutant comprises a mutation within the FVIIIa binding site of FIX.

24. The method of any one of paragraphs 20-23, wherein said Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397.

25. The method of any one of paragraphs 20-24, wherein said Factor IX mutant comprises a mutation at N2, S3, K5, L6, F25, D47, N67, E78, E83, N92, G93, V182, D332, R333, R338, T340, K341, I344, M348, G356, P368, T376, E387, and/or I397.

26. The method of any one of paragraphs 20-25, wherein said Factor IX mutant comprises a mutation at C389, A233, R180, R338, E387, 1397, and/or G93.

27. The method of any one of paragraphs 20-26, wherein said Factor IX mutant comprises a mutation at R338, E387, 1397, and/or G93.

28. The method of any one of paragraphs 20-27, wherein said Factor IX mutant comprises a mutation at R338, E387, and/or I397.

29. The method of any one of paragraphs 20-28, wherein said Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, 1344F, I344N, I344P, I344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T.

30. The method of any one of paragraphs 20-29, wherein said Factor IX mutant comprises a mutation selected from the group consisting of N2I, S3P, K5E, L6S, F25C, F25S, D47E, D47G, N67K, E78K, E83K, N92K, N92S, G93S, V182L, D332Y, R333L, R333P, R333Q, R338P, T340R, K341E, 1344F, 1344N, 1344P, 1344S, I334T, M348I, M348K, G356R, P368H, P368T, T376N, E387A, E387G, E387K, and I397T.

31. The method of any one of paragraphs 20-30, wherein said FIX mutant comprises a mutation selected from the group consisting of C389Y, C389G, C389S, C389R, A233T, R180W, R180Q, R180G, R180P, R180L, R338P, E387A, E387G, E387K, I397T, and G93S.

32. The method of any one of paragraphs 20-31, wherein said Factor IX mutant comprises a mutation selected from the group consisting of R338P, E387K, I397T, and G93S.

33. The method of any one of paragraphs 20-32, wherein said FIX mutant comprises a mutation selected from the group consisting of R338P, E387K, and I397T.

34. The method of any one of paragraphs 20-33, wherein said FVIII mimicking bispecific antibody binds Factor IXa and Factor X.

35. The method of any one of paragraphs 20-34, wherein said FVIII mimicking bispecific antibody is emicizumab.

36. The method of any one of paragraphs 20-35, wherein said FVIII mimicking bispecific antibody is mim8.

### Definitions

Various terms relating to the biological molecules of the present invention are used hereinabove and also throughout the specification and claims.

The singular forms “a,” “an,” and “the” include plural referents unless the context clearly dictates otherwise.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans. A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule (e.g., antigen-binding fragment), and fusions of immunologically active portions of an immunoglobulin molecule.

The term “bispecific” as used herein means the polypeptide (e.g., antibody or antigen binding fragment) is capable of specifically binding at least two target entities.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of a disease or disorder herein may refer to curing, relieving, and/or preventing the disease or disorder, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

The following example is provided to illustrate various embodiments of the present invention. The example is illustrative and are not intended to limit the invention in any way.

### Example

The armamentarium of hemophilia therapy is rapidly expanding (Arruda, et al., Blood 130 (21): 2251-2256). While HA is now frequently treated with the subcutaneously-administered FVIII-mimicking bispecific antibody emicizumab (Young, G., Thromb. Hemostasis (2021) Blood 138 (26) 2750-2751), HB treatment still requires frequent intravenous-administration of FIX protein. The subcutaneous administration has been revolutionary for HA, with most children with HA transitioning to emicizumab for prophylaxis. Emicizumab has an excellent safety record as a monotherapy and is approved for people with hemophilia A of all ages, including infants. Though gene therapy drugs for HB may be approved soon for adults, children with HB under 18 years of age will still require alternative approaches. Thus, there is an urgent need to develop new HB therapies that can be administered subcutaneously. Herein, specific HB-causing FIX variants are identified that can be treated with emicizumab.

At a molecular level, activated FVIII (FVIIIa) serves as an essential cofactor for the serine-protease activated FIX (FIXa) to proteolytically activate factor X (FX), which is the rate limiting step of sustained coagulation. To test whether emicizumab can treat HB caused by FIX variants with dysfunctional FIXa/FVIIIa interactions, three HB-causing FIX variants with amino acid substitutions (R338P, E387K, and I397T) within the FVIIIa-binding site were screened as well as another amino acid substitution (R248Q) outside the FVIIIa-binding site as a negative control. Herein, it is shown that FIX R338P, E387K, and I397T can be rescued with a therapeutic concentration of emicizumab, resulting in FIX activity levels in the mild HB range (>5% normal). In contrast, FIX R248Q does not improve with emicizumab. Furthermore, emicizumab similarly increases the FIX activity in HB patient plasma with the I397T variant to the mild HB range, while it does not change the FIX activity in plasma from a cross-reactive material negative (CRM−) HB patient. These results show that FVIII-mimicking bispecific antibodies improve the bleeding of HB patients specifically with dysfunctional FIXa/FVIIIa interactions. As 50% of severe and moderate HB-causing variants are due to missense mutations, this approach will be applicable to a large number of patients with HB.

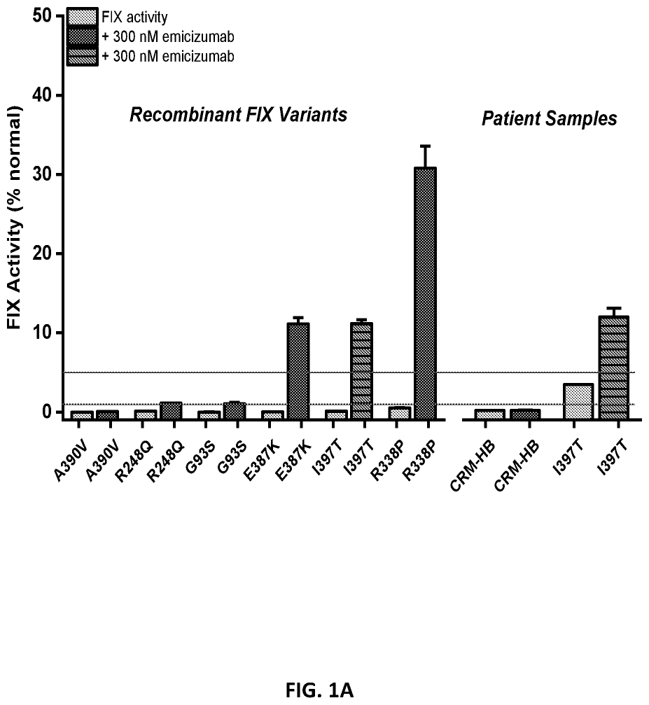

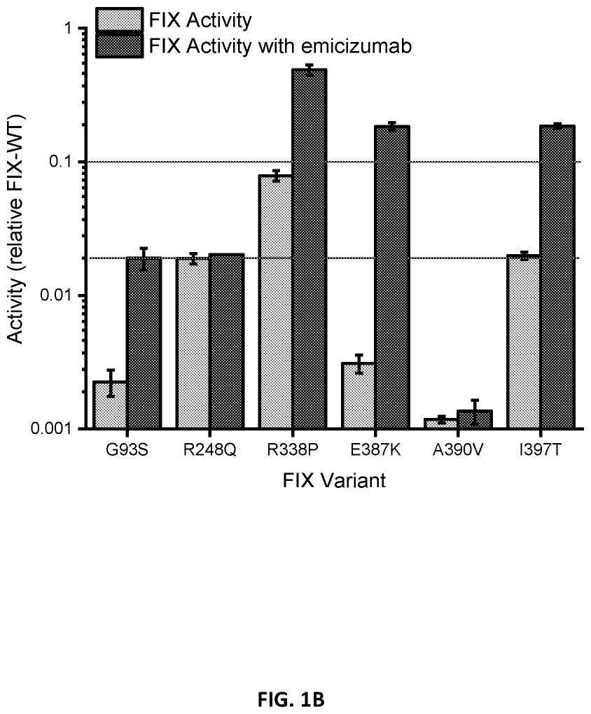

FIG. 1A shows that specific HB-causing FIX variants that have dysfunctional FIXa/FVIIIa interactions (R338P, E387K, and I397T) can be rescued with therapeutic concentrations of emicizumab, resulting in FIX activity levels in the mild HB range (>5% normal). FIX variants were transiently expressed from HEK293 cells and activity was measured using a one-stage activated partial thromboplastin time (aPTT) based clotting assay in FIX-deficient plasma or FIX-deficient plasma with 300 nM emicizumab. In contrast, other HB-causing FIX variants such as A390V and R248Q do not significantly improve with emicizumab. Furthermore, emicizumab similarly increases the FIX activity in HB patient plasma with the I397T variant to the mild HB range, while it does not change the FIX activity in plasma from a cross-reactive material negative (CRM−) HB patient (FIG. 1A). These results show that FVIII-mimicking bispecific antibodies—such as emicizumab—can improve the bleeding of HB patients specifically with dysfunctional FIXa/FVIIIa interactions. These mutations can be identified by structural analysis of the putative FIXa/FVIIIa binding site and then, optionally, tested as in FIG. 1A.

I397T, E387K, R338P, and G93S all substantially increase their activity with the addition of emicizumab, indicating their low activity is secondary to a dysfunctional interaction with FVIIIa and their hemostatic function can be improved with a FVIIIa mimetic. The FIX activity of I397T and E387K increase from the moderate and severe range respectively to the mild range with emicizumab. The FIX activity of R338P increases from the low mild range to a curative level with emicizumab. G93S increases from the severe range into the moderate range. A390V and R248Q do not show an improvement with emicizumab, consistent with their location outside the FVIIIa-binding site and communication wire.

FIG. 1B depicts the variants activity on a log scale. Both G93S and R248Q exhibit approximately 2% activity in the presence of the FVIII-mimetic, but only 0.1% and 2% activity without the FVIIIa-mimetic. In other words, G93S activity increases approximately tenfold, while R248Q activity does not change. Clinically, G93S has a severe phenotype while R248Q is mild.

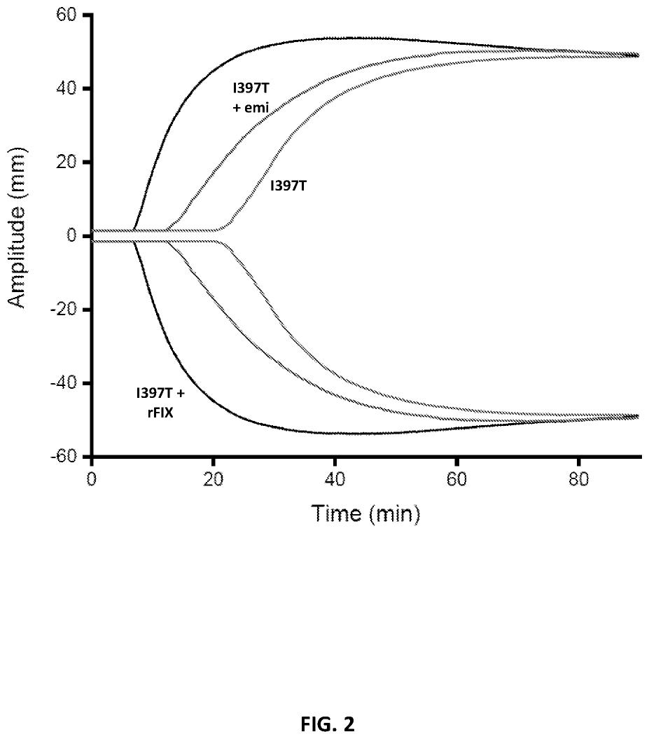

The ability of the FVIII-mimetic emicizumab to improve hemostatic activity of FIX I397T is shown in FIG. 2. Briefly, modified whole blood from a patient with moderate HB due to FIX I397T was evaluated with rotational thromboelastometry (ROTEM). ROTEM evaluates viscoelastic properties during blood clot formation. As seen in FIG. 2, the pro-coagulant activity of FIX I397T substantially increased with the addition of 300 nM emicizumab to over half 100% FIX activity.

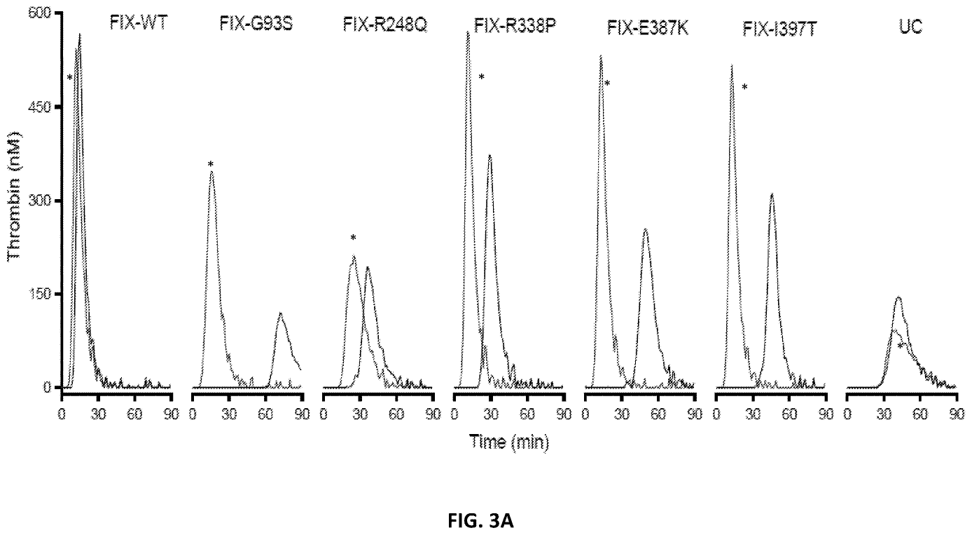

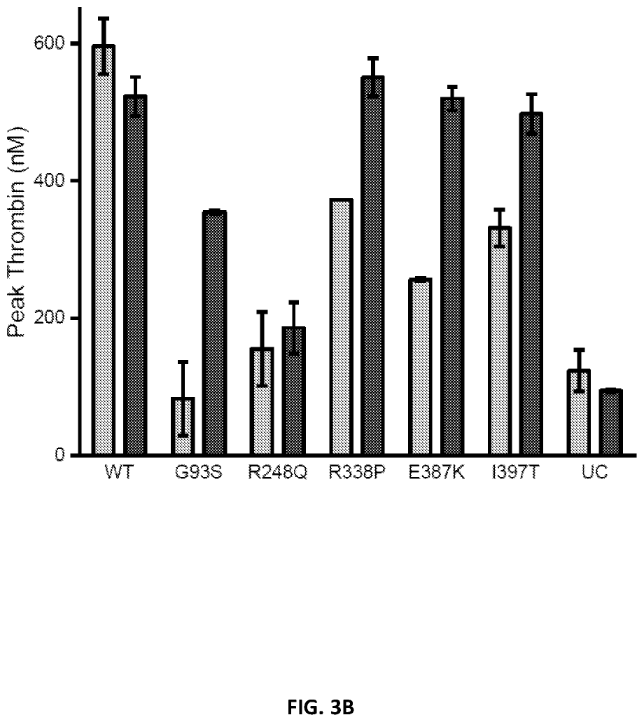

As seen in FIG. 3, the FVIII-mimetic emicizumab improves thrombin generation of dysfunctional HB-causing FIX variants. Recombinant FIX variants causing HB were expressed from mammalian cells and assayed for thrombin generation with and without 300 nM emicizumab. FIG. 3A provides representative thrombograms of FIX-WT and HB-causing FIX variants. FIG. 3B provides a quantification of peak thrombin with a negative control of unconditioned (UC) media.

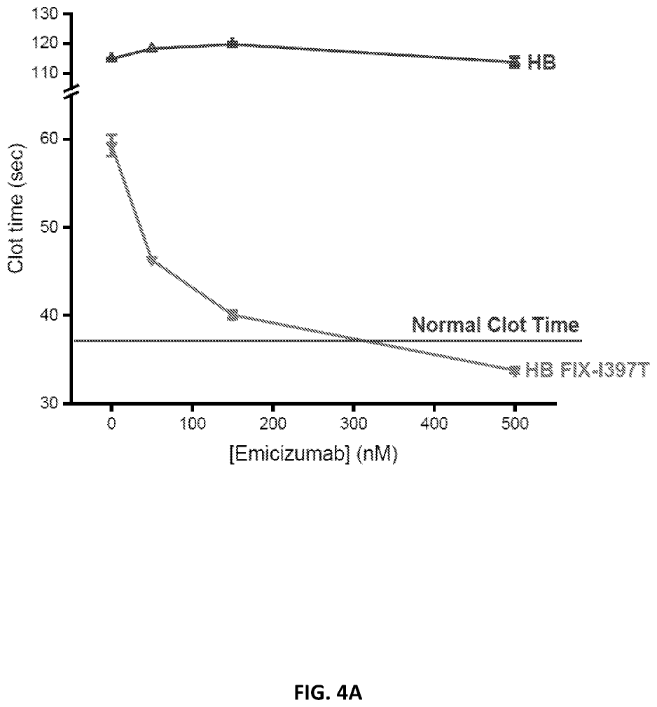

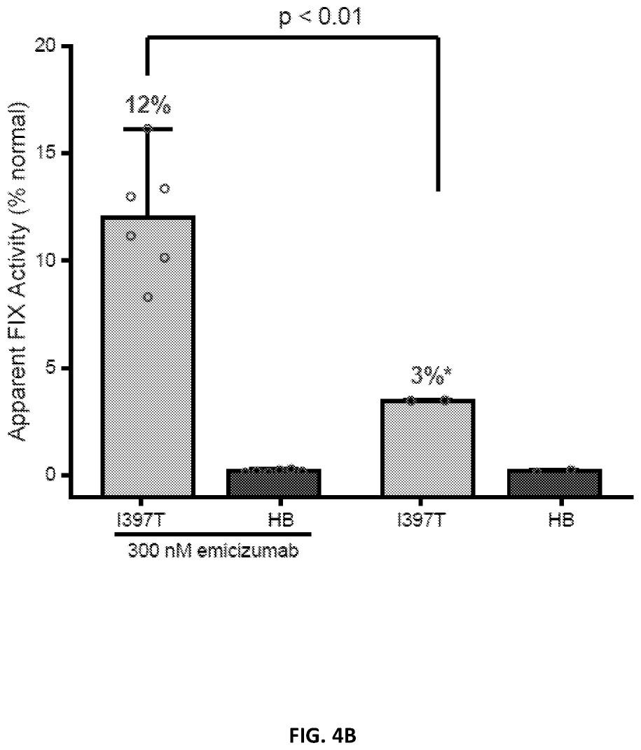

Clotting assays were also performed with the FIX I397T variant. Briefly, the FVIII-mimetic was added to HB FIX I397T or HB plasma and clot times were triggered by addition of aPTT reagent. As seen in FIG. 4A, the addition of emicizumab resulted in reducing the clotting time to normal levels. A one-stage aPTT assay was also used to determine FIX activity in the presence or absence of a FVIII-mimetic (FIG. 4B). Without the addition of the FVIII-mimetic emicizumab, the FIX I397T sample has an activity only 3% of normal, which is consistent with clinical lab measurement. However, with the in vitro addition of 300 nM emicizumab, the FIX activity of FIX I397T increases to 12% of normal.

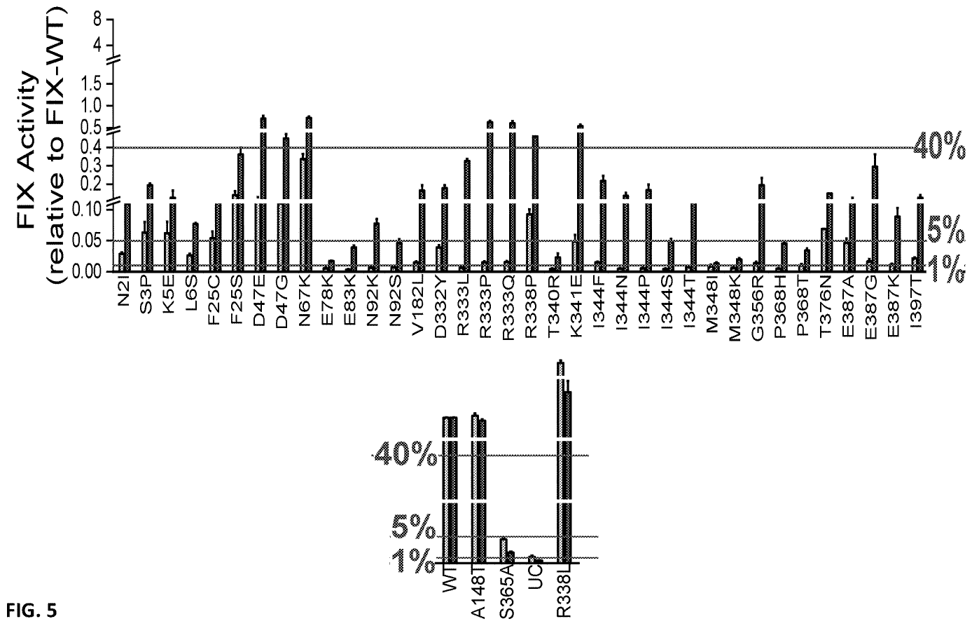

The FIX activity of various other mutants in the presence or absence of emicizumab were also tested (FIG. 5). As seen in FIG. 5, rescuable variants (top) have ≥2 fold increase in FIX activity with the addition of emicizumab. Control FIX variants (FIG. 5, bottom) include wild-type (WT), non-deleterious polymorphism (A148T), catalytically inert (S365A), hyperactive R338L, and unconditioned media (UC).

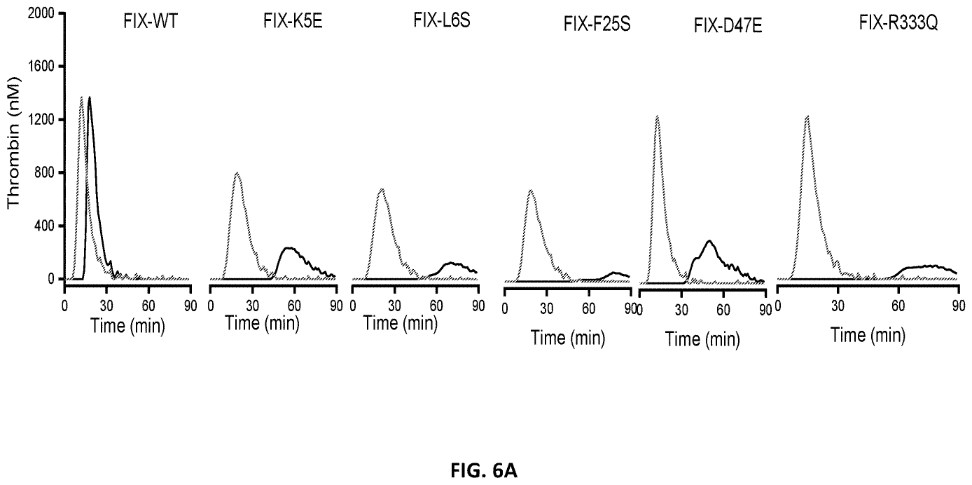

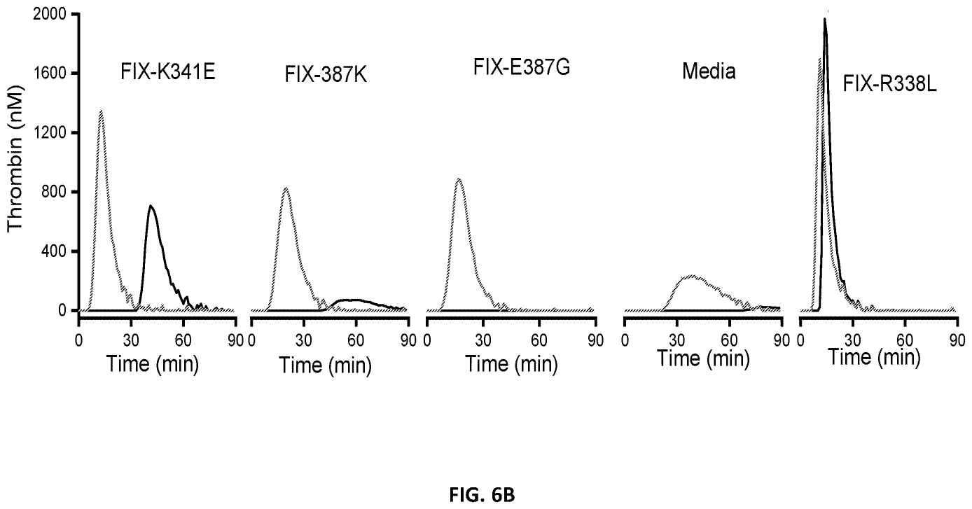

FVIII-mimetic emicizumab also improved thrombin generation by various dysfunctional HB-causing FIX variants (FIGS. 6A and 6B). Thrombin generation was evaluated for rescuable mutants K5E, L6S, F25S, D47E, R333Q, K341E, E387K, and E87G. Media, wild-type (WT), and hyperactive R338L are provided as controls.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of treating or inhibiting hemophilia B in a subject in need thereof comprising administering a therapeutically effective amount of a Factor VIII (FVIII) mimicking bispecific antibody to said subject, wherein said subject comprises a Factor IX (FIX) mutant, and wherein activated FVIII (FVIIIa) has decreased binding affinity for the activated FIX (FIXa) mutant compared to wild-type FIXa and/or the FIXa mutant has decreased FVIIIa-induced allosteric activation compared to wild-type FIXa.

2. The method of claim 1, wherein said FIX mutant comprises a mutation within the FVIIIa binding site of FIX.

3. The method of claim 1, wherein said Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397.

4. The method of claim 1, wherein said Factor IX mutant comprises a mutation at N2, S3, K5, L6, F25, D47, N67, E78, E83, N92, G93, V182, D332, R333, R338, T340, K341, I344, M348, G356, P368, T376, E387, and/or I397.

5. The method of claim 1, wherein said FIX mutant comprises a mutation at C389, A233, R180, R338, E387, I397, and/or G93.

6. The method of claim 1, wherein said FIX mutant comprises a mutation at R338, E387, 1397, and/or G93.

7. The method of claim 1, wherein said FIX mutant comprises a mutation at R338, E387, and/or I397.

8. The method of claim 1, wherein said Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, 1344F, 1344N, 1344P, I344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T.

9. The method of claim 1, wherein said Factor IX mutant comprises a mutation selected from the group consisting of N2I, S3P, K5E, L6S, F25C, F25S, D47E, D47G, N67K, E78K, E83K, N92K, N92S, G93S, V182L, D332Y, R333L, R333P, R333Q, R338P, T340R, K341E, 1344F, 1344N, 1344P, 1344S, I334T, M348I, M348K, G356R, P368H, P368T, T376N, E387A, E387G, E387K, and I397T.

10-13. (canceled)

14. The method of claim 1, further comprising determining said subject comprises said Factor IX mutant prior to administering said FVIII mimicking bispecific antibody.

15. The method of claim 14, wherein determining said subject comprises said Factor IX mutant comprises at least partially sequencing a nucleic acid encoding Factor IX from said subject.

16. The method of claim 14, further comprising obtaining a biological sample from said subject.

17. A method for increasing blood coagulation comprising contacting blood with a Factor VIII (FVIII) mimicking bispecific antibody, wherein said blood comprises a Factor IX (FIX) mutant, and wherein activated FVIII (FVIIIa) has decreased binding affinity for the activated FIX (FIXa) mutant compared to wild-type FIXa and/or the FIXa mutant has decreased FVIIIa-induced allosteric activation compared to wild-type FIXa.

18. The method of claim 17 which is an in vitro method.

19. The method of claim 17, wherein said Factor IX mutant comprises a mutation within the FVIIIa binding site of FIX.

20. The method of claim 17, wherein said Factor IX mutant comprises a mutation at N2, S3, K5, L6, F25, D47, N67, E78, E83, N92, G93, R180, V182, A233, D332, R333, R338, T340, K341, I344, M348, G356, P368, T376, E387, C389, and/or I397.

21. The method of claim 17, wherein said Factor IX mutant comprises a mutation at N2, S3, K5, L6, F25, D47, N67, E78, E83, N92, G93, V182, D332, R333, R338, T340, K341, I344, M348, G356, P368, T376, E387, and/or I397.

22. The method of claim 17, wherein said Factor IX mutant comprises a mutation at C389, A233, R180, R338, E387, 1397, and/or G93.

23. The method of claim 17, wherein said Factor IX mutant comprises a mutation at R338, E387, 1397, and/or G93.

24. The method of claim 17, wherein said Factor IX mutant comprises a mutation at R338, E387, and/or I397.

25. The method of claim 17, wherein said Factor IX mutant comprises a mutation selected from the group consisting of N2D, N2Y, N2I, S3P, K5E, L6S, F25C, F25S, D47N, D47H, D47E, D47G, N67K, E78K, E83K, N92H, N92K, N92S, G93D, G93S, G93R, G93K, G93N, R180W, R180Q, R180G, R180P, R180L, V182L, V182F, V182A, V182G, A233T, D332Y, R333G, R333L, R333P, R333Q, R338P, T340I, T340R, K341N, K341E, 1344F, 1344N, 1344P, I344S, I334T, M348V, M348R, M348I, M348K, G356R, G356E, P368S, P368L, P368R, P368H, P368T, T376N, E387A, E387G, E387K, C389Y, C389G, C389S, C389R, I397L, and I397T.

26. The method of claim 17, wherein said Factor IX mutant comprises a mutation selected from the group consisting of N2I, S3P, K5E, L6S, F25C, F25S, D47E, D47G, N67K, E78K, E83K, N92K, N92S, G93S, V182L, D332Y, R333L, R333P, R333Q, R338P, T340R, K341E, 1344F, 1344N, 1344P, 1344S, I334T, M348I, M348K, G356R, P368H, P368T, T376N, E387A, E387G, E387K, and I397T.

27. The method of claim 17, wherein said Factor IX mutant comprises a mutation selected from the group consisting of R338P, E387K, I397T, and G93S.

28. The method of claim 17, wherein said FVIII mimicking bispecific antibody binds Factor IXa and Factor X.

29. The method of claim 17, wherein said FVIII mimicking bispecific antibody is emicizumab.

30. The method of claim 17, wherein said FVIII mimicking bispecific antibody is mim8.

31. The method of claim 1, wherein said Factor IX mutant comprises a mutation selected from the group consisting of R338P, E387K, I397T, and G93S.

32. The method of claim 1, wherein said FVIII mimicking bispecific antibody binds Factor IXa and Factor X.

33. The method of claim 1, wherein said FVIII mimicking bispecific antibody is emicizumab.

34. The method of claim 1, wherein said FVIII mimicking bispecific antibody is mim8.
